(19) United States
(12) Patent Application Publication
(10) Pub. No.: US 2004/0024452 A1
(43) Pub. Date: Feb. 5, 2004

(54) Valved prostheses with preformed tissue leaflets

(76) Inventors: Steven D. Kruse, Bloomington, MN (US); Matthew F. Ogle, Oronoco, MN (US)

Correspondence Address: ALTERA LAW GROUP, LLC, 6500 CITY WEST PARKWAY, SUITE 100, MINNEAPOLIS, MN, 55344-7704, US

(21) Appl. No.: 10/211,026

(22) Filed: Aug. 2, 2002

(51) Int. Cl. A61F 2/24

(52) U.S. Cl. 623/2.13; 623/2.12; 623/918

(57) ABSTRACT

Valved prostheses are described with crosslinked leaflets. At least one of the leaflets has a shape corresponding to a contoured surface. The leaflets are individually attached to the prostheses. Furthermore, in some embodiments, the leaflets do not comprise native leaflet tissue. Methods for forming tissue heart valve prostheses can comprise assembling a plurality of leaflets configured to open and close the valve in response to pressure differentials. Each of the plurality of leaflets is preformed individually when at least partially crosslinked in contact with a contoured surface. The individual crosslinked leaflets can be selected and matched for assembly into a valve. In general, the tissue, when it is crosslinked, has a size and shape approximately the size of a single human heart valve leaflet.

### FIELD OF THE INVENTION

The invention relates to valved prostheses with leaflets that comprise crosslinked tissue as well as the crosslinked leaflets themselves. In addition, the invention relates to approaches for crosslinking tissue-based leaflets for subsequent assembly into, a valved prosthesis, such as a heart valve prosthesis.

### BACKGROUND OF THE INVENTION

Bioprosthetic heart valves from natural materials were introduced in the early 1960's. Bioprosthetic heart valves typically are derived from pig aortic valves or are manufactured from other biological materials, such as bovine pericardium. Xenograft heart valves are typically fixed with glutaraldehyde or other crosslinking agent prior to implantation to reduce immunological rejection. A crosslinking agent reacts to form covalent bonds with free functional groups in proteins, thereby chemically crosslinking nearby proteins.

The importance of heart valve prostheses with tissue leaflets as replacements for diseased or damaged human heart valves has resulted in a considerable amount of interest in the design, formation and long term performance of these valves. The character of natural tissues poses issues that are not faced with respect to most synthetic materials. Specifically, quality control and uniformity of the raw materials are not as easy to control for natural materials. For example, when assembling bioprosthetic heart valves from segments of tissue, structural irregularities in the tissue can complicate the process and make the process less reproducible. A low level of reproducibility can result in waste and added expense due to discarded prostheses with leaflets that do not display functional properties within appropriate standards.

### SUMMARY OF THE INVENTION

In a first aspect, the invention pertains to a valved prosthesis comprising a plurality of leaflets each of which comprises crosslinked tissue. At least one of the leaflets has a shape corresponding to a contour. The leaflets are individually attached to the prosthesis. In some embodiments, the leaflets comprise pericardium, fascia or a combination thereof and do not comprise native leaflet tissue.

In a further embodiment, the invention pertains to a method for forming a valved prosthesis. The method comprises assembling a plurality of leaflets configured to open and close the valve in response to pressure differentials. The plurality of leaflets has been crosslinked individually in contact with a contoured surface.

In another aspect, the invention pertains to a method for forming a leaflet for a valved prosthesis. The method comprises crosslinking a tissue segment having the approximate size of a single human heart valve leaflet. The crosslinking is performed in contact with a contoured surface.

In an additional aspect, the invention pertains to a tissue segment comprising crosslinked tissue having the shape and size of a single human heart valve leaflet. The leaflets do not comprise native leaflet tissue.

### BRIEF DESCRIPTION OF THE DRAWINGS

FIG. 1 is a side perspective view of a stentless aortic heart valve prosthesis.

FIG. 2 is a side perspective view of a four leaflet, stentless mitral heart valve prosthesis.

FIG. 3 is a side perspective view of a three leaflet stented heart valve prosthesis.

FIG. 4A is a perspective view of a vascular prosthesis.

FIG. 4B is a side view of the vascular prosthesis of FIG. 4A attached to blood vessels.

FIG. 5 is a fragmentary side view of a left ventricular assist device with check valves having tissue leaflets, in which the sides of the inflow and outflow tubes have been cut away to expose the inflow and outflow valves.

FIG. 6 is a perspective view of a mandrel with a curved surface for the crosslinking of a tissue segment to be used as a valve leaflet.

FIG. 7 is a perspective view of an alternative embodiment of a mandrel with a curved surface for crosslinking tissue and an internal network of channels or porous section for supplying a vacuum to hold a tissue segment onto the mandrel surface.

FIG. 8 is a schematic perspective view of a system for crosslinking tissue using a mandrel having a curved surface to immerse a tissue segment on the curved surface in a crosslinking solution.

FIG. 9 is a schematic perspective view of an alternative embodiment of a system for crosslinking tissue using a curved surface in which crosslinking solution is sprayed onto the tissue.

FIG. 10 is a side view of a leaflet section of the prosthesis of FIG. 1.

FIG. 11 is a side view of a post segment of the prosthesis of FIG. 1.

FIG. 12 is a side view of a strip of the prosthesis of FIG. 1.

FIG. 13 is a side perspective view of a stent and a leaflet from the prosthesis of FIG. 3.

FIG. 14 is a side perspective view of the leaflet of FIG. 13 partially attached to the stent.

FIG. 15 is a side perspective view of the stent of FIG. 13 with two leaflets partially attached to the stent.

### DETAILED DESCRIPTION OF THE INVENTION

Improved approaches for forming prosthetic valves with tissue-based leaflets involve separately fixing/crosslinking of a tissue section corresponding to an individual leaflet or a portion of tissue from which an individual leaflet is cut. The crosslinking is performed on a particular contour corresponding to the face of a leaflet. In general, the leaflets are crosslinked in contact with a mandrel or the like that shapes the leaflets to have a selected nonplanar shape. A set of leaflets with similar and/or matched characteristics can be selected for assembly into a multi-leaflet valved prosthesis, such as a heart valve prosthesis. Since the leaflets are selected to have similar/matched characteristics, the valve generally has desirable valve function for long periods of time after implantation with predictable function. By selecting the individual leaflets following crosslinking and before assembly of the valve, the valves can have improved and more reproducible and consistent performance and yield can be improved. In addition, the processing overall can be more efficient.

In general, relevant medical devices are bioprostheses and in particular, valved prostheses, that are formed to mimic a corresponding structure within the body. A bioprosthesis can be used to replace a corresponding native valved structure, such as a heart valve prosthesis. The prosthetic devices can be suitable for long-term implantation within a recipient patient. In embodiments of particular interest, the patient is an animal, preferably a mammal, such as a human. A valved prosthesis generally comprises leaflets where the leaflets move in response to pressure changes to open and close the valve. Tissue leaflets generally have specific mechanical requirements, such as durability, extensibility, flexibility, mechanical strength and tear resistance, for their function within the valved device.

Damaged or diseased native biological valves can be replaced with valved prostheses to restore valve function. For example, heart valve prostheses with tissue leaflets can be designed as a replacement for any heart valve, i.e., an aortic valve, a mitral valve, a tricuspid valve, or a pulmonary valve. In addition, valved prostheses with leaflets formed from selected tissue leaflets can be used for the replacement of vascular valves.

In a prosthetic valve with tissue leaflets, the leaflets flex to open and close the valve. The leaflets are supported by a support structure that includes commissure supports and scallops extending between the commissure supports. The commissure supports hold the ends of the free edge of the leaflets. Commissure supports may or may not extend beyond the attachment points of the leaflet in the flow direction. The attached edge of the leaflet is located along scallops and ends at the commissure support. The attached edges of adjacent leaflets approach each other at the commissure support. The support structure of the valve may comprise a sewing cuff or the like for attachment of the valve to the patient's annulus, to other components of a medical device, or anatomical structure.

In some embodiments, the support structure comprises a rigid component that helps maintain the leaflet function of the valve against the forces opening and closing the valve. Valves with a rigid support structure are termed stented valves, and the rigid support is called a stent. The stent provides a scaffolding for the leaflets. The stent generally is sufficiently rigid such that only the base of the stent is attached to the patient or other device. As a particular example, heart valve stents are used to support leaflet components within a prosthetic heart valve.

In alternative embodiments, the support structure is not sufficiently rigid to maintain the leaflet function of the valve against the forces opening and closing the valve. In these embodiments, the valve is termed stentless. In a stentless valve, the support structure also has commissure supports at which the free edge of the leaflet connects with the support structure, and scallops which support the attached edge of the leaflets. However, in the stentless valve, the support structure is less rigid such that both edges of the support structure, i.e., the inflow edge and the outflow edge, must be secured such as by suturing or other fastening approach to other anatomical structures, such as the wall of a blood vessel, or to other device structures to prevent the valve from collapsing against the fluid pressure. The support structure can be formed from tissue or from other flexible material or materials in a configuration that defines the commissure supports and the scallops or other suitable interface that hold the attached edges of the leaflet.

A tissue-based valve generally includes a plurality of leaflets. Generally, the valves function as one way check valves that open to allow flow in a desired direction and close in response to pressure differentials to limit reverse flow. Thus, during forward blood flow, the leaflets fully open to allow for flow through the valve. In the open position, the free edges of the tissue leaflets form the downstream opening of the valve and generally do not significantly resist forward blood flow.

When the valve closes in response to pressure differentials, the free edges of adjacent leaflets contact in a closed position with the leaflets extending across the lumen of the valve. The contact of adjacent leaflet free edges across the lumen of the valve eliminates or greatly reduces back flow through the valve. The overlapping or contacting portion of the leaflets is referred to as the coaptation region.

In general, bioprosthetic valved prostheses with tissue leaflets can be formed from a natural valve or from a tissue assembled into a valve. For example, fixed porcine heart valves can be used to replace damaged or diseased human heart valves. While entire valves can be used to form prosthetic valves and natural valve leaflets can be extracted to assemble into prosthetic valves, processing of these structures into prosthetic valves can be difficult to perform without damaging the leaflets. Production yields can be low since valves with any flawed or mismatched leaflets cannot be used. Alternatively, porcine heart valve leaflets can be removed from the native valve and assembled into a valved prosthesis, although this approach can face similar limitations as using whole native valves.

The improved approaches described herein are based on the use of tissue types that do not originate from native leaflets or cusps, for example, bovine pericardium and fascia, to form tissue leaflets. For convenience, leaflets and cusps are used interchangeably. In particular, the use of other types of bioprosthetic tissue not originating from valves for use in a bioprosthetic valve provides greater versatility in valve design, a greater availability of materials than with the use of native leaflets and an opportunity to select well matched leaflets to reduce waste. Furthermore, reduced cost and improved yields can result from the formation of leaflets with desired properties from selected tissue materials.

Suitable materials for incorporation into prostheses generally are biocompatible, in that they are non-toxic, non-carcinogenic and do not induce hemolysis or a significant immunological response. Heart valve prostheses formed from tissue generally are non-thrombogenic. Relevant mechanical properties of flexible leaflets include, for example, stiffness, strength, creep, hardness, fatigue resistance and tear resistance.

In general, the tissue for leaflet formation can be xenograft, allograft, autograft, biosynthetic tissue or combinations thereof. The tissue is harvested for processing and may or may not be stored prior to performing the processing. Some preliminary processing can be performed prior to crosslinking the tissue, such as cutting and trimming of the tissue, sterilizing the tissue, associating the tissue with one or more desirable compositions, such as anticalcification agents and growth factors, and the like. After any preliminary processing and/or storage is completed, the tissue is crosslinked, generally in contact with a curved surface. Following crosslinking of the tissue, the tissue can be further processed, which can involve additional chemical and/or mechanical manipulation of the tissue as well as processing the tissue into the desired valve structure.

Crosslinking or fixing tissue can be performed, for example, to mechanically stabilize the tissue, decrease or eliminate antigens and/or terminate enzymatic activity. Xenograft tissue, i.e., tissue transplanted between species, generally is crosslinked to reduce or eliminate immune response. Crosslinked tissue generally refers to tissue that is completely crosslinked in the sense that further contact with a crosslinking agent does not further change measurable attributes of the tissue.

Crosslinking of the tissue involves a chemical crosslinking compound with a plurality of functional groups that bond to the tissue to form a chemically crosslinked material. The chemical crosslinking reaches completion once the crosslinking agent has permeated the tissue and reacted with the accessible binding sites of the tissue.

Once a sufficient level of crosslinking is reached, the mechanical properties of the tissue are generally determined or set. While total (100%) crosslinking is not needed to achieve the desired mechanical properties, some less crosslinked tissue may not have the desired mechanical properties. While the mechanical properties of crosslinked tissue are stabilized, the tissue may gradually change upon exposure to physiological conditions or under inappropriate storage conditions, such as dehydrating conditions for reasonable periods of time. With proper storage, the crosslinked tissue has stable mechanical properties. Since the crosslinked tissue has stable mechanical properties, the mechanical and physical properties of the tissue can be matched, such that the properties of the respective leaflets are within desired tolerances.

If the leaflets are matched within a valve, the leaflets in a closed configuration can have balanced stresses/strains such that the coapting edges of adjacent leaflets form a stable closed configuration. In contrast, leaflets with unmatched properties have a tendency toward prolapse and regurgitation, which can result in more rapid degradation. A crosslinked assembled valve can be evaluated to reject valves in which the leaflets are not appropriately matched. However, this results in considerable waste with respect to disposing of the entire valve. Thus, conventional processing approaches for tissue valve assembly may have a low yield due to mismatched leaflets within the valve.

Tissue properties can be evaluated prior to crosslinking as well as after crosslinking. In evaluating the tissue prior to crosslinking, tissue that does not fall within desired ranges of properties can be discarded or directed to other uses. The evaluation can be based on visual observations and/or particular measurements, as described further below. In addition to using the evaluation of the uncrosslinked tissue for identifying inappropriate tissue, the evaluation of the tissue properties can be used for leaflet matching of the leaflets used in the prosthesis. Thus, the properties of the leaflets/tissue before crosslinking and following crosslinking can be used together for selecting leaflets to be used together.

Following crosslinking of the tissue, processing the tissue into the leaflets can include, for example, cutting the tissue section to an appropriate size and shape (if not done prior to crosslinking), optionally processing the leaflets for additional desired properties, selecting the leaflets to be matched within a single valve and assembling the leaflets together to form the valve structure along with any other appropriate tissue or non-tissue components. The assembled valves are packaged and shipped to health care professionals for implantation into a patient. The tissue leaflets are particularly suitable for forming stented valves, although unstented valves can also be formed.

The evaluation and matching of crosslinked leaflets properties can be performed before or after final cutting and/or before or after any further processing of the tissue. However, selection of matched leaflets generally is performed prior to assembly of the leaflets into the valve. Any additional cutting of the leaflets can be performed to specifications based on the particular valve design and size. The additional processing of the leaflets can involve, for example, treatments with anticalcification agents, growth factors and other desirable property modifiers. The treatment with desirable property modifiers can be performed before or after assembly of the valve.

The selection process involving an evaluation and a matching can be based on criteria related to particular properties of the valve. The selection can be based on evaluations of the tissue before crosslinking and/or after crosslinking. The matching generally is based on features that relate to proper coaptation of the leaflets in the valve. Improved coaptation of the leaflets leads to stable valve function and can lead to long term proper valve operation.

Valved Prostheses

The crosslinked tissue leaflets can be used in various valved prostheses. In particular, tissue leaflets can be used, for example, in artificial hearts, heart valve prostheses, valved vascular prostheses or left ventricular assist devices. Heart valve prostheses with tissue leaflets are suitable for the replacement of damaged or diseased native heart valves. With appropriate sizing and attachment, the tissue-based valves of the present invention are suitable for replacement of any of the heart valves. In general, heart valve prostheses can be designed and constructed with a selected numbers of tissue leaflets, such as two leaflets, three leaflets, four leaflets or more than four leaflets. In appropriate embodiments, the prosthesis may or may not have the same number of leaflets as the natural valve that it is used to replace.

Mammalian veins include valves that assist with blood circulation by limiting the amount of back flow in the veins. Veins collect blood from capillaries and are responsible for returning blood to the heart. Generally, vascular valves are replaced as part of a vascular graft with sections of conduit.

Ventricular assist devices are mechanical pumps that are implanted into a patient to assist their heart. Left ventricular assist devices are generally used to maintain the ventricular pumping function of a patient with a damaged or diseased heart awaiting a heart transplant, although they have also been proposed for longer term use. The pumping function of the heart uses check valves analogous to the valves of a heart chamber. Since blood flows through the pump, the pump components including the check valves should be biocompatible. Thus, prosthetic heart valves are suitable for use as check valves within a ventricular assist device.

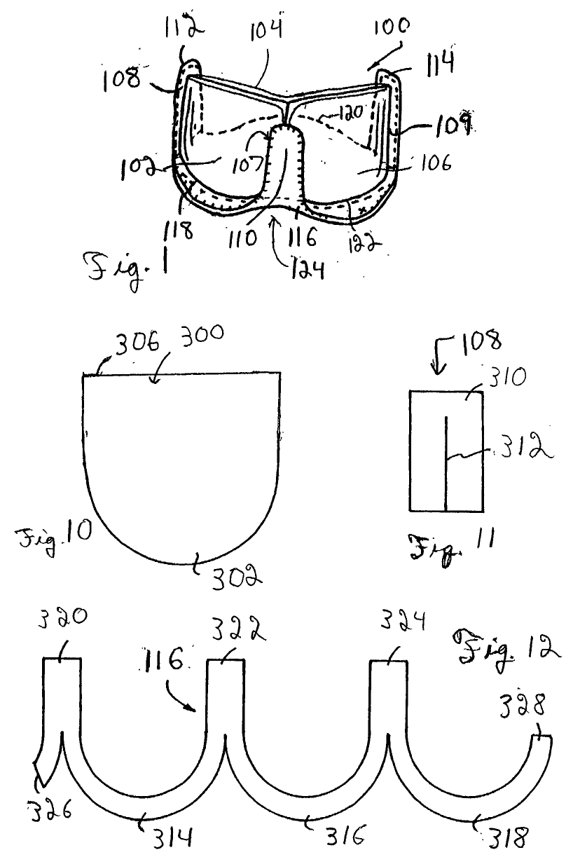

Heart valve prostheses with tissue leaflets can include a stent that serves as a frame for flexible leaflets, or the valve can be stentless, in which a heart valve is implanted utilizing the recipient's native support structure, e.g., the aorta or mitral annulus and chordae. As a particular example of a stentless aortic heart valve prosthesis assembled from oriented tissue elements, heart valve prosthesis 100 has three leaflets 102, 104, 106, as shown in FIG. 1. Leaflets 102, 104, 106 are attached to post segments 107, 108, 109 at commissure posts 110, 112, 114. A strip 116 joins to post segments 107, 108, 109 and leaflets 102, 104, 106 along scallops 118, 120, 122 to form a valve structure with an inflow edge 124 at scallops 118, 120, 122. Heart valve prosthesis 100 can be assembled from leaflets that are crosslinked and selected as described herein.

Another example of a heart valve prosthesis assembled from oriented tissue elements is shown in FIG. 2. A stentless mitral heart valve prosthesis 130 with four leaflets includes a sewing ring 132, and four leaflets 134, 136, 138, 140. Chordae 142 extend from leaflets 134, 136, 138, 140. Chordae 142 and/or associated leaflets can be formed from a single sheet of tissue. Chordae 142 connect with attachment sections 144 for attachment to the patient's papillary muscles upon implantation. An edge 146 of the tissue forming leaflets 134, 136, 138, 140 is stitched between two portions 148, 150 of sewing ring 132 to secure the leaflets to the sewing ring. One or more of leaflets 134, 136, 138, 140 can be crosslinked in contact with a curved surface, as described below. In embodiments of particular interest, each of leaflets 134, 136, 138, 140 are separately crosslinked in contact with a curved surface and selected to match each other in desired properties.

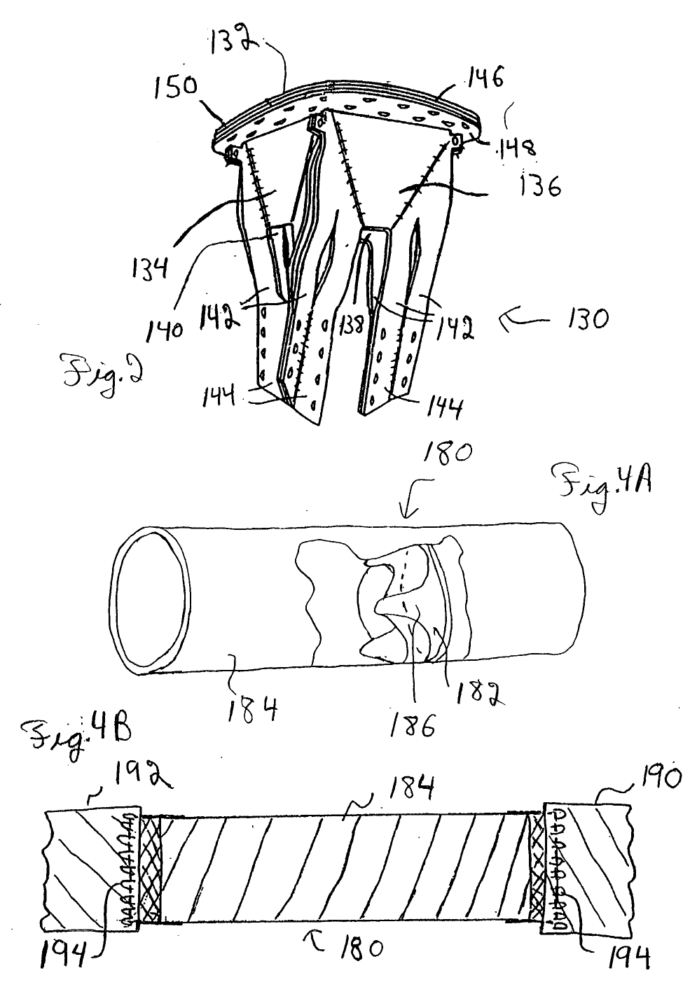

A stented heart valve prosthesis with tissue leaflets is shown in FIG. 3. Stented valve 160 comprises a stent 162, a sewing cuff 164 and three tissue leaflets 166, 168, 170. Stent 162 is made from an appropriate material to prevent the leaflets from collapsing when the valve is closed. The tissue is fastened to the stent to secure the tissue in the valve structure. Sewing cuff 164 facilitates implantation by providing a structure for fastening, such as suturing, the valve to native support structure. In these embodiments, leaflets 166, 168, 170 can be separately crosslinked in contact with a curved surface and matched for similar properties. The curved faces of leaflets 166, 168, 170 meet stent 162 at scallops 172, 174 (third scallop not shown in FIG. 3), respectively. The free edges of leaflets 166, 168, 170 attach to stent 162 at posts 176, 177, 178. Heart valve prosthesis 160 can be used in any valve position within the heart.

The valve prosthesis can be incorporated into a graft for replacement of a venous valve or for the replacement of an aortic or pulmonary heart valve. A vascular graft 180 is shown in a fragmentary view in FIG. 4A. Prosthesis 180 includes a valve 182 in a conduit 184. Support structure/stent 186 can be rigid or flexible, with corresponding appropriate attachment to conduit 184. For example, if support structure/stent 186 is flexible, the support structure is attached along its outflow edge to conduit 184 for support. Conduit 184 can be made from natural materials, such as fixed bovine pericardium, or synthetic materials, such as polymers, for example, polyesters. A side view of vascular graft 180 attached to natural vessel sections 190, 192 is depicted in FIG. 4B. As shown in FIG. 4B, suture 194 is used to secure vascular graft 180 to vessel sections 190, 192, although other fastening approaches can be used.

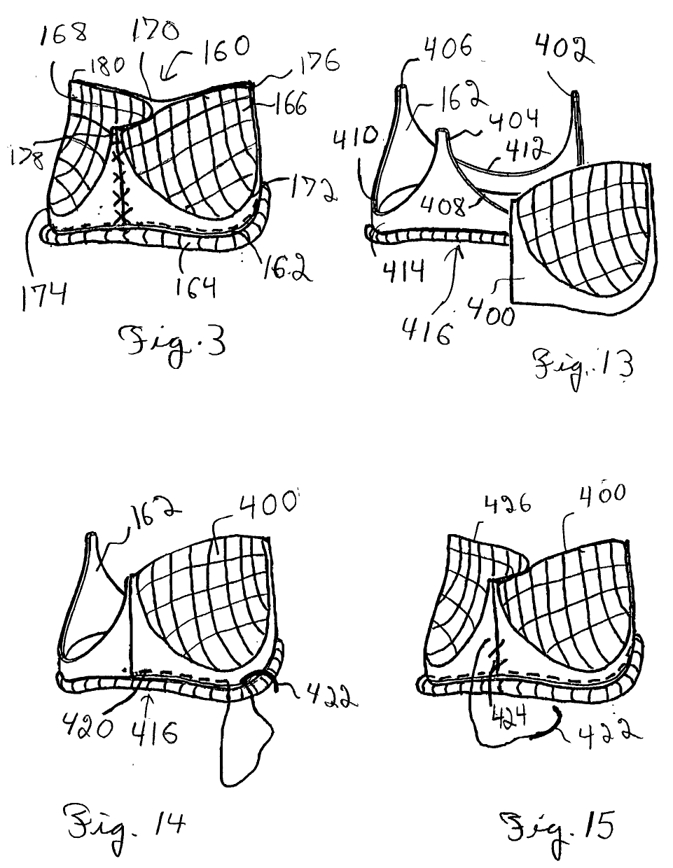

A left ventricular assist device 200 is shown in FIG. 5. Left ventricular assist device 200 includes a drive unit 202, an inflow tube 204, an outflow tube 206 and connection 208. Drive unit 202 includes a pump to provide pulsatile flow from inflow tube 204 to outflow tube 206. Connection 208 provides for electrical or pneumatic control signals to be directed to the drive unit from a controller and power supply, generally external to the patient. Inflow tube 204 includes an inflow valve 210, and outflow tube 206 includes an outflow valve 212. Arrows depict the direction of blood flow through inflow tube 204 and outflow tube 206 as controlled by valves 210, 212. Either one or both of inflow valve 210 and outflow valve 212 can be tissue-based valves as described herein.

Tissue Crosslinking

Tissue comprises a protein-based extracellular matrix that generally comprises structural proteins, such as collagen, elastin and/or glycoproteins, and non-protein components, such as polysaccharides. The tissue can be a natural tissue or a synthetic protein-based matrix. In embodiments of particular interest, the tissue is fully crosslinked or partially crosslinked while conforming with a contoured structure. The contoured structure may or may not have a shape approximating the shape of the leaflet at some point along the valve cycle. The contoured structure can be a mandrel or the like. In some embodiments, tissue is placed in contact with the contoured surface without any additional anchoring other than the surface interactions between the tissue and the surface.

Appropriate bioprosthetic tissue materials can be formed from natural tissues, synthetic tissue matrices and combinations thereof. Synthetic tissue matrices can be formed from extracellular matrix proteins that are combined to form a tissue matrix. Suitable tissues can comprise components of synthetic materials, such as polymers, for example, that have or have had viable cells associated with the synthetic materials, in which the viable cells, when present, formed a proteinaceous extracellular matrix in association with any synthetic materials. Thus, tissue materials generally can have viable cells or protein materials formed from cells that are no longer present, whether or not synthetic materials are present. Suitable polymers, such as polyesters, and extracellular matrix proteins, such as collagen, gelatin, elastin, glycoproteins, silk collagen/elastin and combinations thereof, for incorporation into a synthetic tissue matrix are commercially available.

Natural, i.e. biological, tissue material suitable for leaflet formation includes relatively intact tissue as well as decellularized tissue. Decellularized tissue can be obtained using chemical and/or biological agents, such as hypotonic buffers, hypertonic buffers, surfactants, proteases, nucleases, lipases, other similar agents and combinations thereof, to remove or dismantle cells and cellular structures within the extracellular matrix. These natural tissues generally include collagen-containing material. In particular, natural tissues may be obtained from, for example, native heart valves, portions of native heart valves such as roots and walls, pericardial tissues such as pericardial sacs, amniotic sacs, connective tissues, bypass grafts, skin patches, blood vessels, cartilage, dura mater, skin, fascia, submucosa, such as intestinal submucosa, umbilical tissues, and the like. Natural tissues are derived from a particular animal species, typically mammalian, such as human, bovine, equine, ovine, porcine, seal or kangaroo. These tissues may include a whole organ, a portion of an organ or structural tissue components. Suitable natural tissues include xenografts (i.e., cross species, such as a non-human donor for a human recipient), allografts (i.e., interspecies with a donor of the same species as the recipient) and autografts (i.e., the donor and the recipient being the same individual). Suitable tissue is generally soft tissue.

Synthetic tissue matrices can be formed from structural proteins, e.g., extracellular matrix components, that are assembled into tissue structures, such as sheets or other shapes. For example, purified collagen can be formed into a sheet structure. While purified collagen fibrils may be fragments of native collagen fibrils, purified collagen can be used to form suitable tissue. Other materials, such as other structural proteins, can be combined with the collagen in a synthetic tissue. Other structural proteins for incorporation into synthetic tissue matrices include, for example, elastin, proteoglycans and other glycoproteins. These other structural proteins can modify the properties of the tissue, for example, by introducing added flexibility, elasticity and/or providing the material with a lower friction surface. A section of tissue can have various properties relevant to tissue function as a leaflet for a valve including, for example, thickness, fibrosity, flexibility with respect to deformation or out-of-plane bending generally as well as extensibility with respect to elasticity and in plane expansion and/or compression.

Several layers of tissue can be combined to form a fused tissue structure, which can have improved and/or more uniform properties. These combined layers can comprise fused layers of natural tissue, fused layers of synthetic tissue material or a combination of natural tissue layers and synthetic tissue layers. In particular, for the formation of tissue components for a heart valve, it may be advantageous to form a composite with one or more layers with a high collagen content combined with one or more layers with significant levels of elastin and/or proteoglycans. Natural and/or synthetic tissue layers can be fused together, for example, with lyophilization, adhesives, pressure and/or heat. Chemical crosslinking can also be used to fuse tissue layers together. For chemical crosslinking in contact with the curved surface of a mandrel, some adhesion of the layers prior to placement on the curved surface can reduce any undesired shifting of the layers prior to crosslinking. The use of pressure and/or heat to fuse intestinal submucosa tissue layers is described further in U.S. Pat. No. 5,955,110 to Patel et al., entitled “Multilayered Submucosal Graft Constructs And Methods For Making The Same,” incorporated herein by reference.

As a specific example of forming composites with natural tissue and synthetic tissue matrices, intestinal submucosa can be combined with a synthetic layer comprising collagen, elastin and/or proteoglycans. Intestinal submucosa is a good source of uniform natural tissue with a high collagen content. However, intestinal submucosa alone is more rigid than generally desired for some applications, such as for heart valve leaflets. In addition, intestinal submucosa is thin, such that it would be desirable to combine intestinal submucosa with additional layers, either other layers of natural tissue and/or synthetic materials. Thus, the combination of intestinal submucosa with synthetic layers including compositions that impart added flexibility can result in a composite material that has appropriate overall properties. Specific examples of composites formed with natural tissue, such as intestinal submucosa, and synthetic layers or a plurality of synthetic layers are described further in copending and commonly assigned U.S. patent application Ser. No. 10/027,464 to Kelly et al., entitled “Matrices For Synthetic Tissue,” incorporated herein by reference.

Tissue materials can be fixed by crosslinking. Fixation provides mechanical stabilization, for example, by preventing enzymatic degradation of the tissue and by anchoring the collagen fibrils. Glutaraldehyde, formaldehyde or a combination thereof is typically used for fixation, but other fixatives can be used, such as epoxides, epoxyamines, diimides and other difunctional/polyfunctional aldehydes. In particular, aldehyde functional groups are highly reactive with amine groups in proteins, such as collagen. Epoxyamines are molecules that generally include both an amine moiety (e.g. a primary, secondary, tertiary, or quaternary amine) and an epoxide moiety. The epoxyamine compound can be a monoepoxyamine compound and/or a polyepoxyamine compound. In some embodiments, the epoxyamine compound is a polyepoxyamine compound having at least two epoxide moieties and possibly three or more epoxide moieties. In some embodiments, the polyepoxyamine compound is triglycidylamine (TGA). The use of epoxyamines as crosslinking agents is described further in U.S. Pat. No. 6,391,538 to Vyavahare et al., entitled “Stabilization Of Implantable Bioprosthetic Tissue,” incorporated herein by reference. The crosslinking forms corresponding adducts, such as glutaraldehyde adducts and epoxyamine adducts, of the crosslinking agent with the tissue that have an identifiable chemical structures.

In general, the process to form completely crosslinked tissue requires a significant amount of time, in part, because the crosslinking agent must penetrate through the tissue. Also, the crosslinking process generally reaches a point of completion at which time the properties of the tissue are essentially stable with respect to any additional measurable changes upon further contact with the crosslinking agent. At the point of completion, it is thought that the crosslinking composition forms a stable crosslinked network. Upon completion, the crosslinking is effectively irreversible such that contact over significant periods of time with aqueous solutions without the crosslinking agent present does not result in reversal of the crosslinking and disassembly of the crosslinked network. Presumably, at completion, many, if not all, of the tissue's available functional groups for crosslinking have reacted with a crosslinking agent. Since the formation of a fully crosslinked tissue is a slow process, the degree of crosslinking of the tissue can be selected to range from very low levels to completion of crosslinking.

In embodiments of particular interest, the crosslinking is performed separately with sections of tissue used for the formation of a single leaflet. The tissue sections can be cut to a desired size and shape prior to the crosslinking or final cutting/trimming can be performed after crosslinking. The crosslinking of a tissue section for the formation of a leaflet generally is performed in contact with a curved surface. The crosslinking process tends to fix soft tissue in the shape imposed during the crosslinking process.

Tissue sections of particular interest for forming heart valve prostheses and, in particular, leaflets, generally have a thickness of at least about 50 microns, generally from about 75 microns to about 3 millimeters (mm) and in other embodiments from about 100 microns to about one (1) millimeter. A person of ordinary skill in the art will recognize that additional ranges of thickness within these explicit ranges are contemplated and are within the present disclosure. The size of the leaflet depends on the selected size of the resulting valve. However, the face of a leaflet generally has a chord with a length from about 8 millimeters (mm) to about 32 mm, wherein the chord is the largest edge-to-edge dimension through the center of the tissue section.

Crosslinking Apparatus And Process

In general, tissue segments that correspond to each leaflet are separately crosslinked. To introduce a desired shape to the tissue, a tissue segment generally is crosslinked in contact with a curved surface for at least a portion of the crosslinking period. The tissue overall is contacted with crosslinking solutions for a sufficient period of time to completely crosslink the tissue. The crosslinking solutions can be delivered through one or more apparatuses to perform the crosslinking.

While the tissue sections are generally crosslinked in contact with a curved surface, the tissue sections do not need to be contacting the curved surface for the entire time of the crosslinking. In embodiments of particular interest, the tissue is in contact with the curved surface for sufficient periods of time during the crosslinking such that the tissue conforms to approximately the shape of the curved surface. In other words, following crosslinking, the tissue has approximately the shape of the curved surface under conditions in which the tissue is not subjected to a load. It may be convenient to contact the tissue with the curved surface throughout the crosslinking process.

The tissue can be, for example, immersed in the crosslinking solution during the crosslinking process and/or crosslinked by spraying the crosslinking solution onto the tissue. For embodiments in which the tissue is immersed in a crosslinking solution with a self-polymerizing crosslinking agent, the tissue can be separated from the supply of crosslinking agent by a semipermeable membrane such that higher molecular weight oligomers/polymers of the crosslinking agent are blocked by the semipermeable membrane. Crosslinking using a semipermeable membrane to separate the crosslinking agent supply is described further in U.S. Pat. No. 5,958,669 to Ogle et al., entitled “Apparatus And Method For Crosslinking To Fix Tissue Or Crosslink Molecules To Tissue,” incorporated herein by reference.

The curved contoured surface for supporting the tissue is located on a mandrel or the like. The mandrel is a structure that has a curved surface. The relevant surface has the desired shape, which generally is the desired shape of a tissue leaflet when the leaflet is not under fluidic pressure differentials. The shape can be selected to approximate the leaflet shape of an open valve, of a closed valve or an intermediate position between an open valve and a closed valve. The shape does not need to approximate the configuration of a leaflet at any point along the valve opening and closing cycle. Although, the shape of the mandrel's curved surface does not need to approximate an actual position of the leaflet over the valve cycle, the leaflet generally has a shape such that the crosslinked tissue can freely flex between a fully open configuration with little or no restriction of the forward fluid flow through the valve and a fully closed configuration which blocks most or all back flow through the valve under physiological conditions of fluid pressures. In some embodiments, the mandrel shape corresponds with a leaflet in an almost closed configuration. If the leaflets are crosslinked in an almost closed configuration, the leaflets may have better coaptation in the completed valve. In other embodiments, the contoured surface of the mandrel has a shape roughly corresponding to leaflets that are partially opened to reduce bending stress. In alternative embodiments, the curved surface can have a shape corresponding approximately to an open leaflet configuration or more closely to an open leaflet configuration. Leaflets crosslinked on a surface more closely similar to an open leaflet configuration can result in a valve with a large flow area and a low pressure drop in the open configuration, although leaflets crosslinked in other configuration can have appropriately large flow areas and pressure drop.

An embodiment of a mandrel for tissue crosslinking is shown in FIG. 6. In this embodiment, mandrel 220 has a base 222. Mandrel 220 has a top surface 224 outlined by an edge 226. Generally, the shape of top surface 224 does not matter as long as it does not interfere with the leaflet crosslinking. Similarly, base 222 can have any shape and size that does not interfere with the crosslinking of the tissue. Mandrel 220 comprises a contoured face 228 that provides a surface for the crosslinking of a leaflet. Mandrel 220 can be supported on base 222 during the crosslinking process. As shown in FIG. 6, mandrel 220 has a single contoured surface. However, the mandrel can have additional contours if they are positioned such that the contours do not interfere with the particular surface contacting a particular leaflet. Similarly, if a mandrel includes a plurality of contoured surfaces, a plurality of leaflets can be simultaneously crosslinked with a tissue segment on each contour as long as the tissue segments do not interfere with the crosslinking of the other segments. If the mandrel has a plurality of surfaces, the contoured surfaces are used for crosslinking leaflets individually and may not necessarily all be used simultaneously during the crosslinking process.

A mandrel for the crosslinking process can be made from any material that is inert with respect to the crosslinking solution. In particular, a mandrel can be formed from metal, such as stainless steel, ceramics, or polymers, such as polycarbonates, polyacetal resins, e.g., Delrin®, DuPont, and combinations thereof. The mandrel can be machined, molded or similarly processed to form the desired contours.

Tissue segments can be placed directly onto the contour. Surface tension holds the tissue segments onto the mandrel surface. Due to the compliance of uncrosslinked tissue or partial crosslinked tissue, the tissue segment can adhere well to the contour surface and conform over effectively the entire interface between the contour and tissue segment. However, without more, the tissue segment can shift during the crosslinking process. Movement during the crosslinking process can ruin a tissue segment since the final shape may reflect a hybrid of the multiple positions and since the contouring of the tissue segment may not be properly positioned relative to the edges of the stent.

In other embodiments, the tissue is adhered to the contoured surface mechanically or with a weak biocompatible adhesive or the like. Suitable adhesives include surgical adhesives, such as fibrin glues. In other embodiments, the mandrel includes an interior cavity connected to a low pressure source/vacuum connected to openings along the contoured surface. The vacuum, generally a mild vacuum, holds the tissue onto the mandrel at a particular position where the tissue is placed.

An embodiment of a mandrel with a vacuum is shown in FIG. 7. Mandrel 250 is connected with a vacuum source/pump 252 through tube 254. Vacuum source/pump 252 can be a conventional pump, an aspirator/venturi or the like. Some desirable vacuum sources provide good resolution control with respect to the application of the vacuum. Tube 254 connects to main channel 256. Branch channels 258 are in fluid communication with main channel 256. Branch channels 258 lead to openings 260 on contoured surface 262. The number, size and position of openings 260 can be selected to hold the tissue without damaging the tissue or adversely affecting the crosslinking process. Mandrel 250 includes a base 264 to support mandrel 250 during the crosslinking process. The magnitude of the pressure in channels 256, 258 relative to atmospheric pressure generally is not too high when holding tissue segments since a high vacuum could damage the tissue or affect the crosslinking process and since only small forces are needed to hold the tissue in place. In particular, the pressure in the main channel 256 is generally from about 1 millimeter of Mercury (mmHg) to about 100 mmHg, and in other embodiments from about 3 mmHg to about 20 mmHg. A person of ordinary skill in the art will recognize that additional ranges within these explicit ranges are contemplated and are within the present application. Channels, a portion thereof or the surface connection with the channels can be replaced with a material that is sufficiently porous to provide desired flow through the material. For convenience, the porous material forming a flow channel for suction and the like is still referred to as a channel.

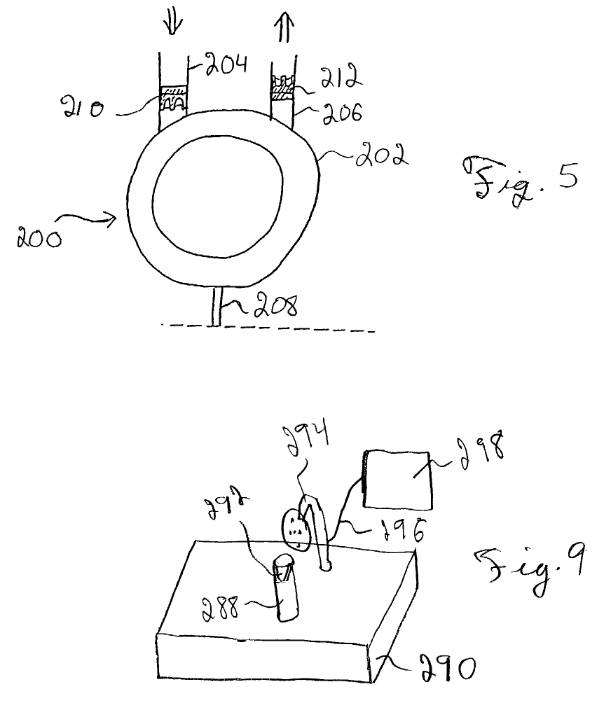

In some embodiments, the tissue on the mandrel is immersed in crosslinking solution. To perform this process, a container is used that holds sufficient amount of crosslinking solution to cover the tissue when the mandrel is positioned within the container. A representative embodiment is shown in FIG. 8. Container 280 contains crosslinking solution 282. Mandrel 284 is within crosslinking solution 282. Tissue segment 286 is placed on a contoured surface of mandrel 284. The tissue can be maintained within the crosslinking solution for the desired length of time. The crosslinking solution can be changed and/or replenished at desired intervals during the crosslinking process. Also, the container can be covered to help prevent contamination and/or to reduce evaporation.

In further embodiments, the tissue on the mandrel is contacted with crosslinking solution by spraying the crosslinking solution onto the tissue. A representative embodiment is shown schematically in FIG. 9. In this embodiment, mandrel 288 is within a tray 290 to collect crosslinking solution directed at tissue element 292 on mandrel 288. Sprayer 294 directs crosslinking solution at tissue element 292. Sprayer 294 can be connected through conduit 296 to source 298 that directs crosslinking solution under pressure to sprayer 294 for directing at tissue element 292. Pressure of the crosslinking solution can be maintained by gravity, a pump or other appropriate approach. Crosslinking solution collected in tray 290 can be recycled back to source 296 with or without additional processing. Mandrel 288 and sprayer 294 can be enclosed to maintain the humidity of tissue element 292. The spray of crosslinking solution can be directed at tissue element 292 continuously, periodically or intermittently with the spraying approach generally being selected to maintain the tissue in a moist state and to achieve the desired tissue crosslinking. A person of ordinary skill in the art can assemble modified spray based crosslinking apparatuses based on this disclosure, as desired.

Additional Tissue Processing

Besides crosslinking, the tissue can be treated with other compounds to modify the tissue properties. Specifically, the tissue can be further modified, for example, to reduce calcification of the tissue following implantation and/or to encourage colonization of the tissue with desired cells. In particular, to encourage colonization with viable cells, the tissue can be treated to reduce or eliminate toxicity associated with aldehyde crosslinking and/or associated with compounds that stimulate the colonization of the tissue by desirable cells.

In some embodiments, tissue crosslinked with dialdehydes or the like can be treated to reduce or eliminate any cytotoxicity. Suitable compounds for reduction of aldehyde cytotoxicity include, for example, amines, such as amino acids, ammonia/ammonium, sulfates, such as thiosulfates and bisulfates, surfactants and combinations thereof. Compositions for the treatment of aldehyde crosslinked tissue to reduce or eliminate cytotoxicity are described further in copending and commonly assigned U.S. patent application, Ser. No. 09/480,437 to Ashworth et al., entitled “Biocompatible Prosthetic Tissue,” incorporated herein by reference.

Generally, any calcification reducing agents would be contacted with the composite matrix following crosslinking, although some calcification reducing agents can be contacted with the tissue prior to crosslinking. Suitable calcification reducing agents include, for example, alcohols, such as ethanol and propylene glycol, detergents (e.g., sodium dodecyl sulfate), toluidine blue, diphosphonates, and multivalent cations, especially Al⁺³, Mg⁺² or Fe⁺³, or corresponding metals that can oxidize to form the multivalent metal cations. The effectiveness of AlCl₃ and FeCl₃ in reducing calcification of crosslinked tissue is described in U.S. Pat. No. 5,368,608 to Levy et al., entitled “Calcification-Resistant Materials and Methods of Making Same Through Use of Multivalent Cations,” incorporated herein by reference. The delivery of anti-calcification agents using microscopic storage structures is described in U.S. Pat. No. 6,193,749 to Schroeder et al., entitled “Calcification Resistant Biomaterials,” incorporated herein by reference.

For some natural tissues, including heart valves, the underlying native tissue includes fibroblast cells within an extracellular matrix. The fibroblast cells produce and maintain the extracellular matrix. The surface of a vascular/cardiovascular tissue has a layer of endothelial cells approximately one cell thick. The endothelial cells provide desirable surface properties to the tissue for blood flow. Specifically, the endothelial cells form a blood contacting surface that is highly non-thrombogenic and blood compatible. Additional treatment of the tissue can involve affiliation of appropriate compounds, especially proteins, with the tissue.

For example, the tissue can be associated with one or more growth factors, such as vascular endothelial growth factor (VEGF) and/or fibroblast growth factor, and/or compounds that attract cell precursors to the tissue, attraction compounds. Suitable colonization stimulating compounds can assist with cellular attachment or the compounds can stimulate cellular proliferation. The compounds are selected based on the desired cell types for colonization of the crosslinked tissue to form a biosynthetic tissue. The use of growth factors, such as VEGF, in the production of prostheses has been described further in copending and commonly assigned U.S. patent application Ser. Nos. 09/014,087 to Carlyle et al., entitled “Prostheses With Associated Growth Factors,” and 09/186,810 to Carlyle et al., entitled “Prostheses With Associated Growth Factors,” both of which are incorporated herein by reference. Fibroblast growth factors refer to a group of proteins that are characterized by the binding of heparin. These proteins have also been called heparin binding growth factors. These proteins strongly stimulate the proliferation of fibroblasts and possibly a variety of other cells of meodermal, ectodermal and endodermal origin.

The use of attraction compounds to associate precursor cells with a substrate is described further in U.S. Pat. No. 6,375,680 to Carlyle et al., entitled “Substrates For Forming Synthetic Tissue,” incorporated herein by reference. The association of a colonization stimulating composition, e.g., a growth factor and/or an attraction compound, with a tissue matrix may involve direct attachment, application of a coating, including an adhesive or binder, or chemical binding, involving a binding agent in addition to the attraction compound/response modifier.

Tissue Selection

In embodiments of particular interest, the leaflets are evaluated following crosslinking and/or prior to crosslinking. The evaluation can lead to rejection of certain tissue sections as well as the matching of tissue sections used for a plurality of leaflets within a single valve. The evaluation of the crosslinked leaflets can involve, for example, a visual inspection, a measurement of thickness variation extensibility and/or flexibility. Similarly, an evaluation of the leaflets prior to crosslinking can involve, for example, a visual inspection, a thickness measurement, extensibility and/or a flexibility measurement. Based on the evaluation process, a plurality of leaflets can be selected to function together within an assembled valve. During the evaluation, the tissue is generally maintained in the same state of hydration to help with proper comparison, and generally the tissue is maintained fully hydrated.

Visual observation of the uncrosslinked tissue can involve an examination to discard any tissue elements that do not meet selected minimum standards. For example, the uncrosslinked tissue can be evaluated for any visible tissue abnormalities that are indicative of tissue that is not suitable for leaflet formation. For example, tissue can be discarded if it is excessively fibrous and/or has excessive fat deposits, excessive prevalence of blood vessels, and/or excessive curvature. The curvature of uncrosslinked tissue can be evaluated by laying the tissue flat and visually examining the tissue. The presence of creases in the flat tissue indicates excessive curvature. Similarly, the amount and size of blood vessels can be evaluated. The visual inspection can also involve identification of thin areas in the tissue, and the rejection of tissue or portions of tissue with visibly thinned sections. Tissue elements with excessive numbers of blood vessels or blood vessels that are too large are discarded or directed to other uses. The flatness of the tissue generally is also evaluated. If the tissue has excessive curvature prior to crosslinking, the tissue may not conform properly to the curved mandrel surface. Thus, if tissue has inappropriate curvature prior to crosslinking, the tissue can be directed to other uses. Additionally or alternatively, the collagen fiber structure can be visibly examined. The amount of fibers visibly present on the tissue surface and the degree and direction of fiber orientation can be evaluated to determine if the tissue is appropriate for leaflets. Some collagen fibers are visible to close inspection with the naked eye. In some embodiments, the tissue is stretched onto a black block under sufficient lighting such that significant features of the tissue generally are visible. Excessive collagen fibers can result in rejection of the tissue for leaflet formation. The degree of orientation of the collagen fibers can be used to orient the tissue appropriately on the mandrel and to match leaflets with tissue with the same degree of collagen fiber orientation.

Flexibility of the tissue prior to crosslinking can be performed, for example, based on a droop test around a rod to evaluate the amount of bending due to gravity. Alternatively or additionally, the flexibility can be evaluated with three points of bending, in which the ends of the tissue are fixed and the tissue is depressed with a probe at the approximate center. The thickness of the tissue prior to crosslinking can be measured at a plurality of points, generally 2 to about 10 points, to evaluate whether or not the tissue has sufficient uniformity and for the matching of leaflets that have comparable thickness such that the leaflets will function similarly and coapt properly in the closed configuration. In some embodiments, the thickness is measured at three points, such as points near the respective attachment point of the free edge and a third point near the center of the leaflet face. The thickness generally is measured using an approach that does not damage the tissue. Suitable measurement approaches include, for example, using a Litematic™ contact measuring device (Mitutoyo, Japan) taken at a 1 gram applied load on wet tissue. The extensibility can be measured by applying a load while holding only the edges of the tissue. The planar deflection of the tissue (uniaxial or biaxial) under the load is related to the extensibility. The properties measured prior to crosslinking can be used for the matching of crosslinked tissue for assembly of the plurality of leaflets within a valve. The evaluation of the properties following crosslinking can be further indicative of excessive shrinkage of the tissue or other unexpected changes in the tissue resulting from the crosslinking.

With respect to the crosslinked leaflet evaluation, a visual evaluation can involve, for example, an examination of the leaflet shape and/or the character of the tissue. With respect to the leaflet shape, the shape of the leaflet can vary from the mandrel shape if the particular leaflet section did not contact the mandrel surface consistently during the crosslinking process. This variation in shape can interfere with desired leaflet function during use. Additionally or alternatively, the appearance of translucent tissue or shiny tissue along all or a portion of the tissue segment can indicate regions of high shrinkage or shrinkage around too tight of a radius. Thus, these tissue sections with undesirable shrinkage and/or variation from the mandrel shape can be discarded.

Furthermore, the properties of the tissue can be examined following crosslinking. For example, the thickness and thickness variation can be measured following crosslinking. Additionally or alternatively, the thickness can be measured to evaluate how the thickness was modified during the crosslinking process. The thickness at several points can be measured using the same techniques as used prior to crosslinking, as described above. The thickness variation can change upon crosslinking. Also, the flexibility can be measured. Flexibility can be measured using a droop test or three point bending test, as described above. However, an alternative flexibility test after crosslinking involves holding the crosslinked leaflet along its periphery corresponding to the fixed edge of the tissue in an assembled valve. Then, a load can be applied to the tissue at one point, such as near the center of the leaflet, or multiple points around the leaflet face, and the deflection of the leaflet can be measured under the load, which can be applied with a contact probe, for example. The deflection can be measured with commercially available tensile testers, such as an Instron tensile tester (Instron Inc., Canton, Mass.) or a MTS instrument. Similarly, the load could be applied with a weight or the like. The extensibility of the crosslinked tissue can be evaluated similarly to the uncrosslinked tissue. While holding the edge(s) of the tissue an in-plane load is applied, and the extension of the tissue can be measured. The extension test can be performed uniaxially or biaxially. Subjective tests can also be performed by trained technicians, for example, by pulling a piece of tissue between their hands to evaluate extensibility of different tissue pieces. Especially before crosslinking, care generally is taken not to deform or damage the tissue during evaluation of the tissue.

One or more of the evaluations can be performed on any particular tissue segment to accept or reject a tissue segment for leaflet use. The evaluation can involve evaluations of suitable tissue segments after crosslinking or prior to crosslinking, although in embodiments of particular interest, the tissue segments are evaluated both prior to crosslinking and after crosslinking.

To match the leaflets, the leaflet thickness, leaflet thickness variation and flexibility can be matched between a plurality of leaflets that are joined within a particular valve. With respect to leaflet thickness, in some embodiments, the average thickness of matched leaflets is selected to vary between leaflets by no more than about 0.002 inches, and the range of thicknesses between two measured points on one leaflets or different leaflets by no more than about 0.004 inches. In general, the thickness of single leaflets can be measured at from about 2 points to about 10 points and in some embodiments at 3 points. With respect to the flexibility, matched leaflets generally are selected to have flexibility that is approximately the same. To evaluate flexibility, the tissue can be placed on a horizontal rod with a diameter, for example, of 0.2-0.3 inches (5.08 mm-76.2 mm) with the center of the disk approximately on the top of the rod. The amount of bending of the disk can be quantified with ratings of 1-3, with 1 corresponding to flexible with an angle from about 0-30 degrees, 2 corresponding with medium flexibility angles about 30-60 degrees and 3 corresponding to stiff with an angle from about 60-90 degrees. In performing the selection, one or more parameters can be the basis of the matching.

Assembly of Medical Devices

The tissue elements can be assembled into a valve following matching of leaflets for the valve. A leaflet support structure provides the framework for the support of the leaflets. The non-leaflet components of a valved medical device can incorporate one or more tissue elements and, optionally, synthetic materials. Tissue elements incorporated into the non-leaflet components of the medical device may or may not be from different tissue sources and or treated differently with respect to crosslinking and/or other additional treatments. Since the crosslinking of the tissue generally determines the orientation of the leaflet in the final valve, the tissue segments can be appropriately mounted in the valve structure to have appropriate leaflet coaptation.

The leaflets can be cut to a desired shape and/or size before or after crosslinking. Generally, the tissue segment is cut to approximately the desired shape and size prior to crosslinking such that any extra tissue does not significantly interfere with the crosslinking process. However, some trimming and cutting can be performed after crosslinking is completed. Similarly, the cutting of the tissue can be performed before or after treatment with any biologically active compositions for modifying the tissue properties. Furthermore, the assembly of the prosthesis components, if required, also can be performed before or after treatment of the tissue with any biologically active compositions.

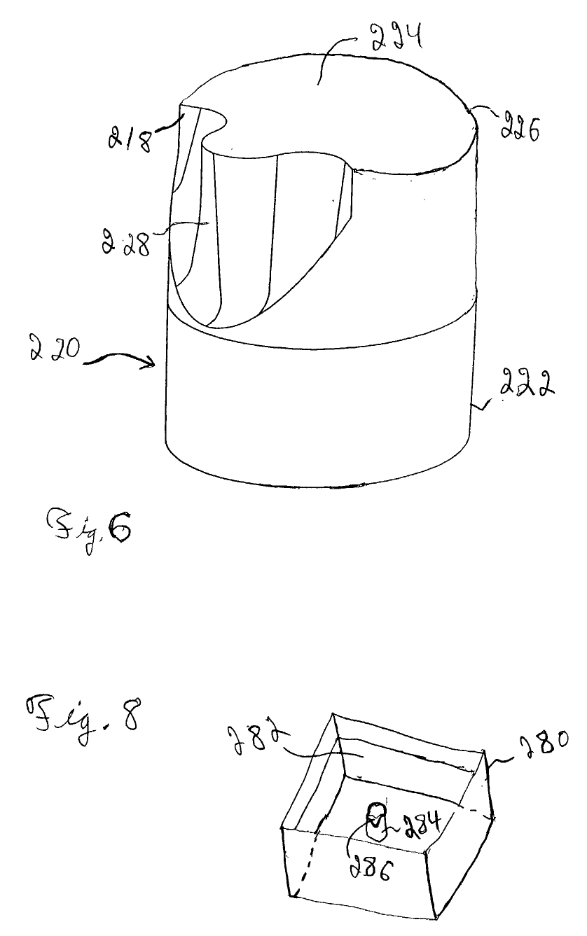

As an example of the assembly process, the heart valve prosthesis of FIG. 1 can be assembled from three structures of tissue portions, as shown in FIGS. 10-12. Referring to FIG. 10, three leaflet segments 300 are used to form valve 100 (FIG. 1). One leaflet segment 300 forms each of the leaflets 102, 104, 106 in the completed valve 100. As shown in FIG. 10, leaflet segment 300 is not crosslinked, but leaflets segment 300 is cut to the desired size and shape. In embodiments of particular interest, each leaflet segment is crosslinked on a mandrel to form a curved tissue segment and the three leaflets matched, as described above. Each leaflet segment 300 includes a rounded portion 302 and a free edge 306.

Referring to FIG. 11, post segments 108 include rectangular tissue segments 310 with a slit 312. Slit 312 is placed over two adjacent leaflets with ears 304 of the two leaflets joined at post segment 108. Once the three leaflets are attached with three post segments 108, free edges 306 of the leaflets extend between post segments 108.

Referring to FIG. 12, strip 116 includes curved scalloped sections 314, 316, 318 joined by post sections 320, 322, 324. Scalloped sections 314, 316, 318 are joined to the three respective rounded portions 302 of the three leaflets segments 300. Once joined to the leaflet segments 300, scalloped sections 314, 316, 318 form inflow edge 124 of the valve. Post sections 320, 322, 324 join with post segments 108. Thus, leaflet segments 300 are secured along all of their edges except for free edges 306. Ends 326, 328 of strip 116 are secured along a leaflet segment such that strip 116 is attached along the circumference of valve 100. Aortic valve prosthesis 100 can be implanted into a patient with a single suture line for faster implantation. The tissue sections can be attached, for example, with suture, adhesives, staples or the like. In alternative embodiments, the valve is assembled without post segments 108 with strip 116 holding the leaflets in place. Similarly, the four-leaflet heart valve prosthesis of FIG. 2 can be assembled from four tissue components that are joined together to form the valve. Assembly of a similar valve prosthesis is described U.S. Pat. No. 5,415,667 to Frater, entitled “Mitral Heart Valve Replacement,” incorporated herein by reference.

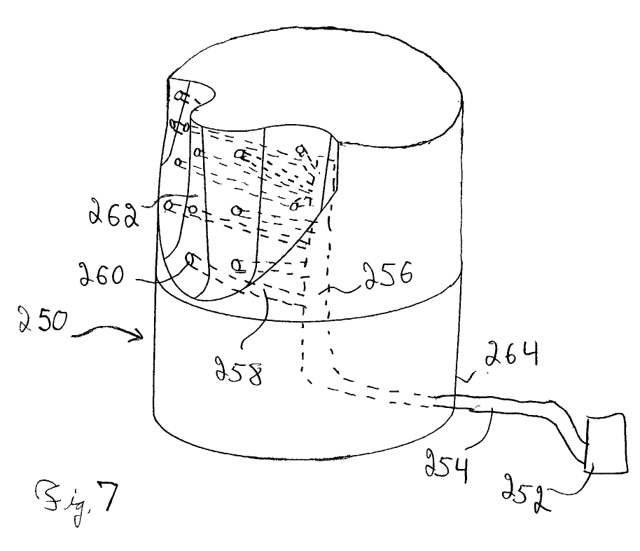

The stented valve of FIG. 3 can be assembled from a stent 162 and three tissue segments, with one segment for each leaflet. The three leaflets can selected to have matched properties as described above, such that the leaflets have proper coaptation and performance. Stent 162 and one tissue segment 400 are shown in FIG. 13. Stent 162 has three commissure posts 402, 404, 406 and three scallops 408, 410, 412 between the commissure posts that together form a band 414 at the inflow edge 416. Referring to FIG. 14, a tissue segment 400 can be initially sutured, stapled, secured with an adhesive or otherwise fastened along the lower edge of the tissue segment toward the inflow edge 416 of the valve. As shown in FIG. 14, suture line 420 is stitched with a suture needle 422, although other fastening approaches can be used. After two adjacent tissue segments are secured, a suture line or other fastening approach can be used to secure the tissue segments along a commissure post 404. Referring to FIG. 15, a suture line 424 is shown partially formed along a commissure post. As shown in FIGS. 13-15, tissue segments 400, 426 are contoured by crosslinking the tissue on a curved surface of a mandrel prior to attachment to stent 162.

Along with tissue components, the medical devices can also comprise one or more other biocompatible materials, such as polymers, ceramics and metals. For example, stents and the like are generally formed from non-tissue materials. Appropriate ceramics include, without limitation, hydroxyapatite, alumina and pyrolytic carbon. Biocompatible metals include, for example, titanium, titanium alloys, cobalt, stainless steel, nickel, iron alloys, cobalt alloys, such as Elgiloy®, a cobalt-chromium-nickel alloy, MP35N, a nickel-cobalt-chromium-molybdenum alloy, and Nitinol®, a nickel-titanium alloy.

Polymeric materials can be fabricated from synthetic polymers as well as purified biological polymers. Appropriate synthetic materials include hydrogels and other synthetic materials that cannot withstand severe dehydration. Suitable polymers include bioresorbable polymers that are gradually resorbed after implantation within a patient.

Appropriate synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, ethylene vinyl acetates, polysulfones, nitrocelluloses and similar copolymers. Bioresorbable synthetic polymers can also be used such as dextran, hydroxyethyl starch, derivatives of gelatin, polyvinylpyrrolidone, polyvinyl alcohol, poly[N-(2-hydroxypropyl) methacrylamide], poly(hydroxy acids), poly(epsilon-caprolactone), polylactic acid, polyglycolic acid, poly(dimethyl glycolic acid), poly(hydroxy butyrate), and similar copolymers. These synthetic polymeric materials can be formed into fibers or yams and then can be woven or knitted into a mesh to form a matrix or substrate. Alternatively, the synthetic polymer materials can be extruded, molded or cast into appropriate forms.

Biological polymers can be naturally occurring or produced in vitro by fermentation and the like or by recombinant genetic engineering. Purified biological polymers can be appropriately formed into a substrate by techniques such as weaving, knitting, casting, molding, extrusion, cellular alignment and magnetic alignment. Suitable biological polymers include, without limitation, collagen, elastin, silk, keratin, gelatin, polyamino acids, polysaccharides (e.g., cellulose and starch) and copolymers thereof.

Colonization of the Tissue with Cells

In some embodiments, the aligned tissue is suitable for in vivo or in vitro affiliation of cells with the tissue, although the tissue can be useful in some applications even if no cell colonization takes place. For in vivo affiliation with cells following implantation, the tissue is assembled into a desired medical device and implanted. If the tissue is prepared for cell colonization, the tissue is suitable seeding ground for cell colonization by cells that are circulating in the patient's fluids. Thus, circulating cells of the patient affiliate with the tissue and can form a repopulated biosynthetic tissue material.

In vitro cell colonization is performed in a cell culture system. With in vitro colonization, the cell colonization can be performed prior to or after assembly of the tissue into a valved medical device. In some embodiments, a combination of in vivo and in vitro cell colonization can be used. For example, inner layers of the tissue can be colonized by selected cells in vitro to provide cell proliferation within the tissue while additional cell types can be colonized in vivo.

The in vitro affiliation of cells with the tissue involves placing the aligned tissue into a cell culture system with the desired cells. The cell culture system can include one or more different cell types. Alternatively, the tissue can be transferred sequentially to different cell culture systems, each with one or more cell types, for the association of the tissue with multiple cell types. To reduce the possibility of transplant rejection, the mammalian cells used for in vitro colonization preferably are autologous cells, i.e., cells from the ultimate recipient. In vitro affiliation of cells with tissue can be performed at hospitals where the patient's cells can be removed for use in a cell culture system. Appropriate cells include, for example, endothelial cells, fibroblast cells, corresponding precursor cells and combinations thereof. Association of endothelial cells is particularly appropriate in the production of prostheses that replace structures that naturally have an endothelial or epithelial cell lining, such as vascular components, cardiovascular structures, portions of the lymphatic system, uterine tissue or retinal tissue. Fibroblasts are capable of a variety of different functions depending on their association with a specific tissue. Myofibroblasts are fibroblasts that express relatively more contractile proteins such as myosin and actin.

The cells can be harvested from the patient's blood or bone marrow. Alternatively, suitable cells could be harvested from, for example, adipose tissue of the patient. The harvesting process can involve liposuction followed by collagenase digestion and purification of microvascular endothelial cells. A suitable process is described further in S. K. Williams, “Endothelial Cell Transplantation,” Cell Transplantation 4:401-410 (1995), incorporated herein by reference, and in U.S. Pat. Nos. 4,883755, 5,372,945 and 5,628,781, all three incorporated herein by reference.

Purified endothelial cells can be suspended in an appropriate growth media such as M199E (e.g., Sigma Cell Culture, St. Louis, Mo.) with the addition of autologous serum. Other cell types can be suspended similarly. The harvested cells can be contacted with the aligned tissue in a cell culture system to associate the cells with the tissue. Thus, a biosynthetic tissue is formed based on cells from the patient prior to implantation.

A tissue can be incubated in a stirred cell suspension for a period of hours to days to allow for cell seeding. Cell seeding provides random attachment of cells that can proliferate to line the surface of the prosthetic substrate either before or after implantation into the patient. Alternatively, the tissue can be incubated under a pressure gradient for a period of minutes to promote cell sodding. A suitable method for cell sodding can be adapted from a procedure described for vascular grafts in the S. K. Williams article, supra.

In addition, the tissue can be placed in a culture system where the patient's cells, such as endothelial cells, are allowed to migrate onto the surface of the prosthetic substrate from adjacent tissue culture surfaces. If either attachment or migration of endothelial cells is performed under conditions involving physiological shear stress, then the endothelial cells colonizing the surface of the tissue may express appropriate adhesion proteins that allow the cells to adhere more tenaciously following implantation.

Storage and use of Tissue and Tissue-Based Devices

The crosslinked tissue can be stored prior to or after formation into a valved prosthesis. Suitable storage techniques generally have a low risk of microbial contamination. For example, the tissue can be stored in a sealed container with sterile buffer, saline solution and/or an antimicrobial agent, such as glutaraldehyde or alcohol.

For distribution, the valved medical devices/prostheses can be placed in sealed and sterile containers for shipping. To ensure maintenance of acceptable levels of sterility, the tissue can be transferred to the sterile container using accepted aseptic protocols. The containers can be dated such that the date reflects an appropriate advisable storage time.

The containers generally are packaged with instructions for the use of the medical devices along with desired and/or required labels. The containers are distributed to health care professionals for surgical implantation of the medical device, e.g., prostheses. The implantation is performed by a qualified health care professional. The surgical implantation generally involves the replacement or supplementation of damaged tissue with the prosthesis.

### EXAMPLE

Tri-Leaflet Valve with Mandrel Crosslinked Leaflets

This example demonstrates the performance of a valve formed from three tissue leaflets that were individually crosslinked in contact with the contoured surface of a mandrel.

Bovine pericardium was obtained from an FDA approved slaughterhouse. The tissue is chilled and the tissue is removed from the heart at the slaughterhouse. Additionally, large deposits of fat are removed from the tissue. The tissue was stored in chilled saline and transported to a manufacturing facility for further processing. Tissue was visually inspected for areas within each pericardial sac that meet the visual inspection criteria of no excessive blood vessels, curvature, fibrosity or thinning and no other deformations. Acceptable regions of a pericardial sac were cut from the remainder of the sac, further cleaned of fat or other deposits, and stored in chilled saline. When processing was continued, rectangular areas slightly larger than a leaflet were cut out from the stored acceptable regions. The rectangular areas were measured for thickness, flexibility and extensibility. The thickness was measured with a Mitutoyo measurement apparatus. Flexibility and extensibility were subjective measurements of feel. The rectangular pieces were positioned onto a single-contour mandrel made from Delrin® polymer. The tissue was held in place by surface tension.

The crosslinking of the tissue was performed with a 0.5% (weight percent stock solution diluted on a volume-per-volume basis) citrate-buffered glutaraldehyde solution. The tissue on the mandrel was dipped into the glutaraldehyde solution, and then the tissue was smoothed onto the contour to eliminate any air bubbles between the leaflet and the contoured surface. The mandrel was allowed to sit in air for five minutes prior to further contact with the crosslinking solution. Then, the dipping process was repeated for a total of three dips into the solution to keep the tissue from dehydrating. After sufficient repeats of the dipping process to allow the tissue to begin to conform to the contour, the mandrel with the tissue was placed into the glutaraldehyde solution and the crosslinking was allowed to go to completion. One batch of 12 leaflets were processed similarly. The leaflets were treated with ethanol and a sterilization solution. The ethanol treatment followed the procedure in U.S. Pat. No. 5,746,775 to Levy et al., entitled “Method Of Making Calcification-Resistant Bioprosthetic Tissue,” incorporated herein by reference.

After crosslinking, the leaflets were examined to select three leaflets for incorporation into the valve. Specifically, the leaflets were matched from the batch for closest thickness, flexibility, general shape and extensibility. Three closely matched leaflets were individually mounted onto a 25 mm stent (corresponding to the outside diameter of the leaflets when on the stent) formed from a titanium-aluminum-vanadium alloy. Maximum thickness variation across the three leaflets was less than about 0.004 inches. The shape of the stent is similar to that as shown in FIG. 13. Each leaflet was sutured around the fixed edge of the leaflet.

The valve was flow tested at physiological conditions, specifically, 70 beats per minute, 5 liters per minute cardiac output, 100 mmHg mean pressure and a 35% systolic duration. Under these conditions, the valve had a 2.66 cm² effective orifice area. Subsequently, the valve was tested on accelerated life testing equipment to 300 million cycles. No visible tissue damage or change in valve function or performance was present. Also, all three leaflets functioned approximately the same.

The embodiments above are intended to be illustrative and not limiting. Additional embodiments are within the claims. Although the present invention has been described with reference to preferred embodiments, workers skilled in the art will recognize that changes may be made in form and detail without departing from the spirit and scope of the invention.

What we claim is:

1. A valved prosthesis comprising a plurality of leaflets each of which comprises crosslinked tissue having a shape corresponding to a contoured surface, the leaflets being individually attached to the prosthesis.

2. The valved prosthesis of claim 1 wherein the leaflets do not comprise native leaflet tissue from a heart valve.

3. The valved prosthesis of claim 1 wherein the tissue comprises pericardium.

4. The valved prosthesis of claim 1 wherein the tissue comprises fascia.

5. The valved prosthesis of claim 1 wherein the tissue comprises a material selected from the group consisting of purified collagen, elastin and proteoglycans.

6. The valved prosthesis of claim 1 wherein the tissue comprise multiple layers with different compositions from each other.

7. The valved prosthesis of claim 1 wherein the crosslinked tissue comprises glutaraldehyde adducts.

8. The valved prosthesis of claim 7 wherein the tissue is treated to reduce the cytotoxicity of the glutaraldhyde crosslinked tissue.

9. The valved prosthesis of claim 1 wherein the crosslinked tissue comprises epoxyamine adducts.

10. The valved prosthesis of claim 1 wherein the prosthesis comprises three leaflets.

11. The valved prosthesis of claim 1 further comprising a stent.

12. The valved prosthesis of claim 1 wherein the valve is stentless.

13. The valved prosthesis of claim 12 wherein the valve further comprises chordae.

14. The valved prosthesis of claim 1 wherein the leaflets have a shape approximating native leaflets at a point along the cycle of opening and closing the valve.

15. The valved prosthesis of claim 1 wherein the leaflets have a shape corresponding with leaflets of a partially open valve.

16. The valved prosthesis of claim 1 wherein the leaflets have a shape approximating a closed configuration for the valve.

17. The valved prosthesis of claim 1 further comprising an anticalcification agent.

18. The valved prosthesis of claim 17 wherein the anticalcification agent comprises ethanol or propylene glycol.

19. The valved prosthesis of claim 1 wherein the leaflets are assembled into the valve with suture.

20. The valved prosthesis of claim 1 wherein the leaflets are assembled into the valve with adhesive.

21. A method for forming a valved prosthesis, the method comprising assembling a plurality of leaflets configured to open and close the valve in response to pressure differentials, wherein each leaflet is preformed when crosslinked individually in contact with a contoured surface.

22. The method of claim 21 wherein the leaflets are crosslinked by immersing the leaflets in a crosslinking solution.

23. The method of claims 21 wherein the crosslinking comprises spraying the crosslinking solution onto the leaflets.

24. The method of claim 21 wherein the leaflets are contacted with the contoured surface until they are completely crosslinked.

25. The method of claim 21 wherein the leaflets are contacted with the contoured surface for a portion of the crosslinking period.

26. The method of claim 21 wherein the leaflets are crosslinked with glutaraldehyde.

27. The method of claim 21 wherein the leaflets are crosslinked with epoxyamine.

28. The method of claim 21 wherein the leaflets following crosslinking have been selected to have matched properties.

29. The method of claim 28 wherein the selection is based at least in part on the shape of the crosslinked leaflets corresponding approximately to the shape of the contoured surface.

30. The method of claim 28 wherein the selection is based at least in part on the thickness and thickness variation of the crosslinked leaflets.

31. The method of claim 28 wherein the selection is based at least in part on the flexibility of the crosslinked leaflets.

32. The method of claim 28 wherein the selection is based at least in part on the extensibility of the crosslinked leaflets.

33. The method of claim 21 wherein the leaflets are evaluated prior to crosslinking.

34. A method for forming a leaflet for a valved prosthesis, the method comprising crosslinking a tissue segment having the approximate size of a single human heart valve leaflet wherein the crosslinking is performed in contact with a contoured surface.

35. The method of claim 34 wherein the tissue segment is approximately planar prior to crosslinking.

36. The method of claim 34 wherein the tissue segment comprises pericardium.

37. The method of claim 34 wherein the crosslinking is performed with a crosslinking agent comprising glutaraldehyde.

38. The method of claim 34 wherein the crosslinking comprises immersing the mandrel and tissue segment in a crosslinking agent.

39. The method of claim 34 wherein the crosslinking comprises spraying the crosslinking solution onto the leaflets.

40. The method of claim 31 wherein the contoured surface is located on a mandrel.

41. The method of claim 40 wherein the mandrel comprises a channel within the mandrel connecting to at least one opening on the curved surface and to a vacuum source and wherein the pressure in the channel holds the tissue segment in place on the curved surface during at least a portion of the crosslinking process.

42. The method of claim 41 wherein the vacuum source comprises a pump.

43. The method of claim 42 wherein the vacuum source comprises a venturi.

44. A tissue segment comprising crosslinked tissue having the shape and size of a single human heart valve leaflet wherein the leaflets do not comprise native leaflet tissue.

45. The tissue segment of claim 44 wherein the tissue comprises pericardium.

46. The tissue segment of claim 44 wherein the crosslinked tissue comprises glutaraldehyde adducts.

47. The tissue segment of claim 46 wherein the tissue is free of detectable cytotoxicity.

48. The tissue segment of claim 44 having a shape of a leaflet corresponding with partially open valve when not under stress.

49. A valve structure comprising a plurality of leaflets of claim 44.
